(19) United States
(12) Patent Application Publication
(10) Pub. No.: US 2008/0132717 A1
(43) Pub. Date: Jun. 5, 2008

(54) Propylene oxide system

(76) Inventors: Bernard Cooker, Malvern, PA (US); John H. Speidel, Media, PA (US)

Correspondence Address: LyondellBasell Industries, 3801 WEST CHESTER PIKE, NEWTOWN SQUARE, PA, 19073, US

(21) Appl. No.: 11/633,196

(22) Filed: Dec. 4, 2006

(51) Int. Cl. C07D 301/06

(52) U.S. Cl. 549/524

(57) ABSTRACT

In a propylene oxide process wherein propylene is oxidized in a reaction mixture comprised of a solid catalyst slurried in a solvent and wherein propane and propylene are separated by distillation in a C₃ splitter, the improvement of removing exothermic heat of reaction by indirect heat exchange with propane, separating the heated propane into a liquid and a vapor fraction and heating the C₃ splitter distillation with the said propane vapor.

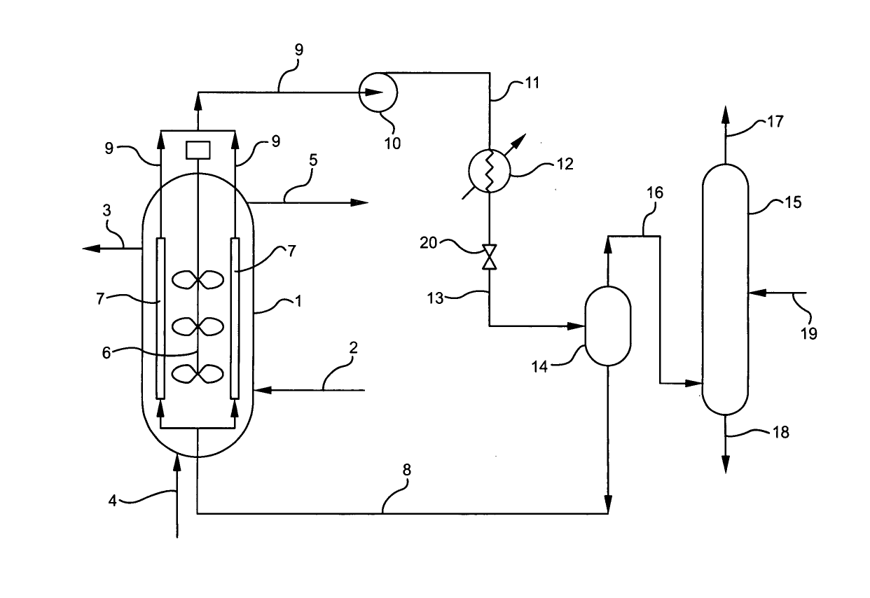

### FIELD OF THE INVENTION

The present invention relates to an integrated reactor and C₃ splitter operation for the production of propylene oxide by direct oxidation.

### DESCRIPTION OF THE PRIOR ART

It is known to produce propylene oxide by reaction of propylene, oxygen and hydrogen using a noble metal containing titanium silicalite solid catalyst. See, for example, Japanese Kokai No. 4-352771 and various other references such as U.S. Pat. Nos. 6,710,194, 7,026,492, 7,057,056, and 6,960,671.

Generally, the solid catalyst is maintained as a slurry in an appropriate solvent such as methanol or methanol and water during reaction of the reactant gases.

The reaction by which propylene oxide is formed is exothermic and a reaction system for carrying out the reaction, of necessity, requires the effective removal of the reaction heat.

Various systems have been proposed for removing the heat of reaction such as packed reactors, the use of internal indirect heat exchange with chilled water, and the like. However, such prior systems have not been entirely satisfactory with regard to ease and expense of operation.

### BRIEF DESCRIPTION OF THE INVENTION

In accordance with the present invention, an improved system is provided for the production of propylene oxide by catalytic reaction in a reaction zone of propylene, oxygen and hydrogen in a reaction medium comprised of a slurry of solid catalyst in an appropriate liquid. There is provided in the reaction zone indirect heat exchange means in which a liquid comprised of propane is heated by indirect heat exchange with the reaction mixture slurry whereby the heat of the exothermic reaction to form propylene oxide is transferred to the propane. In especially preferred operation, the heat so-removed by indirect heat exchange is used to provide distillation heat to a C₃ splitter thereby to effect distillation separation of propane and propylene components of the reaction system.

### DESCRIPTION OF THE DRAWING

The attached drawing illustrates in diagrammatic form a preferred practice of the invention.

### DETAILED DESCRIPTION

Referring to the attached drawing, reactor 1 is a reaction vessel suitable for carrying out the reaction of propylene, oxygen and hydrogen in a reaction liquid containing a slurried solid catalyst for the reaction such as a palladium containing TS-1 catalyst.

A slurry of catalyst particles in suitable liquid solvent such as a methanol/water mixture is introduced via line 2 and a slurry containing catalyst as well as product propylene oxide is removed via line 3 and passes to suitable separation and product recovery means (not shown).

The propylene, hydrogen and oxygen reactants are introduced to reactor 1 via line 4 and vapor products are removed via line 5 for separation and recovery (not shown).

Reactor 1 is provided with stirring means 6 which provide appropriate agitation to the reaction mixture slurry contained therein.

An essential feature of the present invention is the provision of indirect heat transfer elements 7 within the reactor adapted for the indirect transfer of exothermic heat of reaction to a fluid passing within elements 7.

With regard to the heat transfer elements 7 located within reactor 1, any of the known type elements including cooling coils can be used. Preferred, however, is the provision of plate coils or the equivalent as are well known in the art.

Sufficient of elements 7 are provided to remove the exothermic heat of reaction generated by the formation of propylene oxide in reactor 1.

In processes for the production of propylene oxide, propane is normally present in significant quantities as an impurity with the feed propylene and as an undesired by-product which is formed in the system. A necessary item of equipment in such processes is a C₃ splitter or distillation column wherein the separation of propylene and propane by distillation is accomplished, the propylene usually being recycled and the net propane being purged to prevent build-up.

In practice of the present invention, the fluid used in elements 7 to remove the reaction exotherm is comprised of propane, preferably propane from the C₃ splitter which is used to separate propylene and propane. The propane has its origin as an impurity in the feed propylene introduced into reactor 1 via line 4 and as an impurity formed in reactor 1 during propylene oxide formation.

In an especially preferred practice, and as shown in the drawing, heat of reaction is removed from the propane coolant by compression and indirect heat exchange. The propane coolant is removed from the reactor as a vapor and this vapor is compressed essentially to the point where substantial condensation of the propane takes place upon cooling eg. to 68° C. in a cooling water heat exchanger. The temperature of the propane is substantially raised during compression and the compressed propane is cooled and condensed as by indirect heat exchange with cooling water while the higher pressure is maintained. The cooled propane can be subjected to a pressure let-down and liquid and vapor fractions separated. This procedure is simple and convenient and does not require use of a chilled water stream; cooling tower water at 90 F can be used.

As shown in the drawing, the heat transfer stream comprised of liquid propane passes to the heat transfer elements 7 such as plate coils in reactor 1 wherein it absorbs the reaction exotherm. The heated propane suitably now as a vapor, passes via lines 9 to compressor 10 wherein the propane is compressed to a pressure sufficiently high such that the propane upon passing via line 11 to cooler 12 is cooled therein by conventional cooling water and liquified at the compression pressure.

After cooling in heat exchanger 12, the saturated liquid propane at high pressure passes through valve or orifice 20 whereby the pressure is let down and the mixture is flashed to a vapor/liquid mixture. Vapor and liquid are separated in separator 14, liquid propane is passed via line 8 to reactor 1 to provide the heat transfer liquid.

An important aspect of preferred practice of the invention is the use of propane vapor from separator 14 as a heat source for the separation of propylene and propane in C₃ splitter 15. Flashed propane vapor passes from separator 14 to C₃ splitter 15 via line 16. The propane vapor provides heat to distillation column or splitter 15 which is necessary for the overhead distillation of a propylene stream, this being separated via line 17, for example for recycle to reactor 1. The bottoms propane stream is removed via line 18 and can be further treated as desired (not shown).

A C₃ stream comprised of propane and propylene can be fed to splitter 15 via line 19 and separated into its components by conventional operations.

The propane heat removal agent as used herein generally contains at least 75 mol % propane, preferably at least 90 mol % propane. Small amounts of C₄ alkanes, e.g. 2 mol % or less, preferably 1 mol % or less can be present. Propylene can be contained in the heat removal stream in amount up to 25 mol %, preferably 10 mol % or less.

The following example will serve to illustrate practice of the invention.

### EXAMPLE 1

Referring the accompanying drawing, reactor 1 is a continuous reactor containing a particulate catalytic solid, consisting of titanium silicalite, palladium and inert binders. The reactor also contains a methanol/water mixture in which the catalyst is suspended through mechanical agitation via agitator 6. The catalyst is 10 wt % in the slurry and the reactor operates at a slurry process temperature of 50 C (122 F). The reactor has an inside diameter of 20 feet and a straight side length of 60 feet, containing 101,717 gallons of slurry. The reaction between the hydrogen, oxygen and propylene fed via line 4 to produce propylene oxide and coproduct water produces 58 million Btu/hr, which is removed through plate coils 7 which are arrayed radially and vertically in the reactor, between the agitators and the vessel wall. The plate coils are separated by equal angular spacing azimuthally around the vessel. Reaction conditions employed are those which are conventional for the reaction, e.g. a temperature of 50° C. and pressure of 300 psig.

Boiling liquid propane cooling fluid comprised by weight of 96% propane and 4% propylene flows via line 8 into the base of each plate coil, providing a high cooling fluid heat transfer coefficient of 500 Btu/hr ft² F, instead of the value of 150 to 200 Btu/hr ft² F which is obtained from conventional cooling water. The heat transfer area of the plate coils is 3,118 ft². The boiling liquid propane is at 60 F and 110 psia. The temperature driving force between the reactor process slurry, at 122 F, and the boiling liquid propane, at 60 F, is 62 F or 34 C. This is clearly superior to the temperature driving force of 32 F or 18C, which is obtained from using Texas Gulf Coast cooling tower water, which is at 90 F or 32 C.

The 3118 ft² heat transfer area of the plate coils, with an overall heat transfer coefficient of propane at 300 Btu/hr ft², is a fraction of what is required for tower cooling water, which would be 9063 ft² when using cooling water at 90 F.

The boiling propane coolant provides a better cooling fluid side heat transfer coefficient and a doubled overall temperature driving force to remove the heat from the process slurry to the cooling liquid. The propane vapor which is the product of boiling liquid in the reactor plate coils 7 passes via line 9 at 37,419 lb/hr (latent heat of vaporization=155 Btu/lb) to compressor 10 with a feed temperature of 60 F and a feed pressure of approximately 100 psia. The compressor raises the pressure of the propane vapor in an approximately isentropic manner to a discharge pressure of 350 psia and a discharge temperature of 155 F, 68 C. The slightly superheated vapor passes via line 11 to cooler 12 wherein it is cooled and condensed, with minimal pressure drop, at 350 psia, in a shell and tube heat exchanger, with tower cooling water, at a temperature of 90 F.

The resulting saturated liquid flow from the vapor condenser 12 passes via line 13 and is let down in pressure across nozzle 20, from 350 psia to 110 psia, in an approximately adiabatic manner. The resulting vapor/liquid mixture is at 60 F and it is separated in flash vessel 14 into a saturated liquid propane stream which is recycled via line 8 as the cooling medium in the reactor, as described above, and a saturated vapor stream. The latter is conveyed via line 16 to the base of the propane/propylene splitter 15, where it provides heat as reboiler vapor feed.

C₃ splitter 15 is operated in conventional fashion except for provision of heat via the propane vapor stream. Propylene is removed via line 17 at 6° C. and 85 psig and can be used further in propylene oxide production.

Bottoms liquid propane stream is removed via line 18 at 16° C. and 95 psig.

Advantages of practice of the present invention as compared to conventional procedures include the following:

1. Avoidance of expensive chilled water as a cooling medium.

2. Use of a stream for reactor cooling which would normally require heat for propylene recovery, which is a necessary part of the plant design in any event.

3. Use of boiling fluid heat transfer medium in the reactor cooling heat exchangers, improving the overall heat transfer coefficient and reducing the required heat transfer area.

4. Reduced capital cost for heat exchange surface.

5. Improved slurry agitation in the reactor per unit power input, through reduced volume of reactor internals and less resistance to agitation.

6. Improved heat transfer integration and improved thermodynamic efficiency of the plant heat transfer process.

7. Reduced capital cost for reactors, since larger reactors can be utilized, versus multiple smaller reactors, which have reached their size limit in terms of fitting sufficient heat transfer surface into the vessel.

A comparison of the system of the present invention with a comparable one using Texas Gulf Coast cooling water showing the important advantages of the invention is given in the following Table 1.

TABLE 1
Comparison of Texas Gulf Coast Cooling Water and Boiling Propane

| | Texas Gulf Coast cooling | |
|---|---|---|
| Mode | tower water | Boiling propane |
| Heat exchange duty | 58 × 10⁶ | 58 × 10⁶ |
| (Btu/hr) | | |
| Reactor T (F.) | 122 | 122 |
| Cooling fluid T (F.) | 90 | 60 |
| Delta T (F.) | 32 | 62 |
| Cooling fluid heat transfer | 300 | 500 |
| coefficient (Btu/hr ft² F.) | | |
| Overall heat transfer | 200 | 300 |
| coefficient, U (Btu/hr ft² F.) | | |
| Heat exchange area (ft²) | 9063 | 3118 |

We claim:

1. In a process wherein propylene oxide is formed by oxidation of propylene in a reaction mixture comprised of a slurry containing solid oxidation catalyst and wherein propylene and propane are separated by distillation in a C₃ splitter into a propylene vapor stream and a liquid stream comprised of propane, the improvement which comprises removing exothermic heat from propylene oxide formation by indirect heat exchange with said liquid stream comprised of propane.

2. In a process wherein propylene oxide is formed by oxidation of propylene in a reaction mixture comprised of a slurry containing solid oxidation catalyst and wherein propylene and propane are separated by distillation in a C₃ splitter into a propylene vapor stream and a liquid stream comprised of propane, the improvement which comprises passing said liquid stream comprised of propane to indirect heat exchange with the slurry reaction mixture thereby to remove the exothermic heat of reaction, separating the heated propane into a vapor fraction and a liquid fraction, and passing the propane vapor fraction to the C₃ splitter to provide distillation heat therein.

3. The process of claim 1 wherein the said liquid stream comprised of propane comprises at least 75 mol % propane.

4. The process of claim 1 wherein the said liquid stream comprised of propane comprises at least 90 mol % propane
